(19) United States
(12) Patent Application Publication
(10) Pub. No.: US 2004/0101888 A1
(43) Pub. Date: May 27, 2004

(54) Vector system used in screening active substances

(75) Inventors: Cornelis Calkhoven, Berlin (DE); Achim Leutz, Schonwalde (DE)

Correspondence Address: BRUCE LONDA, NORRIS, MCLAUGHLIN & MARCUS, P.A., 220 EAST 42ND STREET, 30TH FLOOR, NEW YORK, NY, 10017, US

(73) Assignee: Max-Delbruck-Centrum Fur Molekulare Medizin, Berlin (DE)

(21) Appl. No.: 10/631,636

(22) Filed: Jul. 31, 2003

Related U.S. Application Data
(63) Continuation-in-part of application No. PCT/DE02/00367, filed on Feb. 1, 2002.

(30) Foreign Application Priority Data
Feb. 3, 2001 (DE) 101 04 952.8

(51) Int. Cl. C12Q 1/68; C12N 15/00

(52) U.S. Cl. 435/6; 435/320.1

(57) ABSTRACT

The invention relates to a vector system for use in screening active substances that influence translation in the cell. The inventive system is suitable for use in the pharmaceutical industry, in medicine and in molecular biology. The vector system consists of control elements of translation that substantially lie 5′ from the detection cassette, but optionally also 3′ from or within the detection cassette, a detection cassette that encodes two or more protein reporter constructs in different reading frames, said reporter constructs containing signal sequences for the release from the cell and various antigenic sites allowing immobilization and differential detection of reporter proteins.

This application is a continuation-in-part of PCT Application No. PCT/DE02/00367, filed Feb. 1, 2002.

The invention relates to a vector system for use in screening active substances that influence translation in the cell. The fields of application of this invention include the pharmaceutical industry, medicine and molecular biology.

The control of messenger RNA translation to form proteins is an important biological-regulatory process, the deregulation of which is the cause of many diseases. There are already some papers dealing with the pathology of regulation of protein translation in the cell.

For example, increased expression of the eukaryotic initiation factor of translation elF-4E has been found in intestinal carcinoma (Rosenwald et al., 1999; Oncogene 18, 2507). This initiation factor binds the 5′-situated CAP structure of eukaryotic mRNAs, thereby playing a crucial role in initiation of translation. As previously demonstrated, overexpression of elF-4E results in transformation of normal rodent cells into tumor cells and leads to an enhancement of transformation parameters in human cells. On the other hand, elimination of elF-4E suppresses transformation by the ras oncogene, suggesting a fundamental role of translational regulation in carcinogenesis. In this context, the mTOR kinase, which assumes a fundamental function in translational regulation, recently was found to play a key role in the transformation by the oncogenes P3K and Akt (Aoki et al., 2001; PNAS 98, 136). Apart from tumorigenesis, the regulation of translation also play a role in metabolic syndromes. For example, mutations in the 2AK3 gene were identified as a cause in the autosomal-recessive “Wolcolt-Rallison” syndrome (Delepine et al., 2000; Nat. Gen. 25, 406). The 2AK3 gene encodes a kinase which effects phosphorylation of the elF2alpha initiation factor of translation. A number. of other functions of translational control in metabolism, in embryo development, in cell differentiation and infective defense are well-known at present and have been reviewed in the recently published book “Translational Control of Gene Expression” (ed. by Sonenberg, Hershey and Mathews, Cold Spring Harbor Laboratory Press, CSH, N.Y., 2000; ISSN 0270-1847; 39).

The invention is based on the object of developing a vector system allowing immediate observation of translational processes in cells.

The invention is carried out according to the main claim, the subclaims representing preferred embodiments.

### BRIEF DESCRIPTION OF THE DRAWINGS

FIG. 1 Diagram of a vector, the resultant transcript and reporter proteins obtained: by translation of the transcript.

FIG. 2 Diagram of a vector as in FIG. 1, further including a cysteine-cassette that directs the synthesis of a ³⁵S-Cys rich region of the reporter proteins.

The central issue of the invention is a new recombinant reporter plasmid construct (vector system) which will be characterized in more detail below. It consists of a number of modules. These modules are used i) as control elements influencing the initiation of translation or the function of ribosomes, ii) to detect regulatory processes and effect internal balance (e.g. transcription vs. translation), iii) for easier measurement with high sample flow rates (secretion of translational products from cells and immobilization on a micro-scale, purification and detection using antigenic tags).

1. The construct includes control elements of translation. These elements are situated essentially 5′ from the detection cassette (see below), but can also be situated 3′ from or within the detection cassette. The control elements control the differential expression of different protein reporter constructs encoded in the detection cassette.

2. The construct includes a detection cassette. Said detection cassette encodes two or more protein reporter constructs in the same or different reading frames. Differential initiation of translation results in a shift of the reporter amounts, thus reflecting differential translation. In contrast, regulatory processes resulting in a general modification of translation give rise to a uniform shift of reporter expression. Both types of regulation can be included in a calculation and combined in mathematical models, thereby allowing computer-controlled evaluation of the results and automation of the method.

3. The reporter constructs include signal sequences for the release from the cell. Consequently, reporter proteins can be determined either in cell lysates or in the supernatant. This latter property is particularly useful in high-throughput analysis of potential active substances.

4. The reporter constructs include different antigenic sites allowing immobilization and differential detection of reporter proteins. One antigenic site is used to immobilize the reporter proteins, and another antigenic site, different in each reporter protein, is used for differential reporter detection (see Example 1).

Using this recombinant reporter plasmid construct of modular structure, the translational control reporter system (TCRS), substances are investigated for their activity of affecting processes of differential initiation of translation, as well as diverse processes during translation. The TCRS construct is incorporated in cells in a transient or stable fashion. Thereafter, the cells are exposed to substances, and the resulting reporter amounts coming from the construct are determined and compared. The data obtained are used to elucidate processes of translational control.

The system of the invention aids to provide a basis for rapid detection and optimization of active substances that influence translational control processes and can be used in pharmacological correction of diseases. Moreover, the system is suitable for scientific analysis of processes of translational regulation.

With reference to the examples, the invention will be illustrated in more detail below.

### EXAMPLE 1

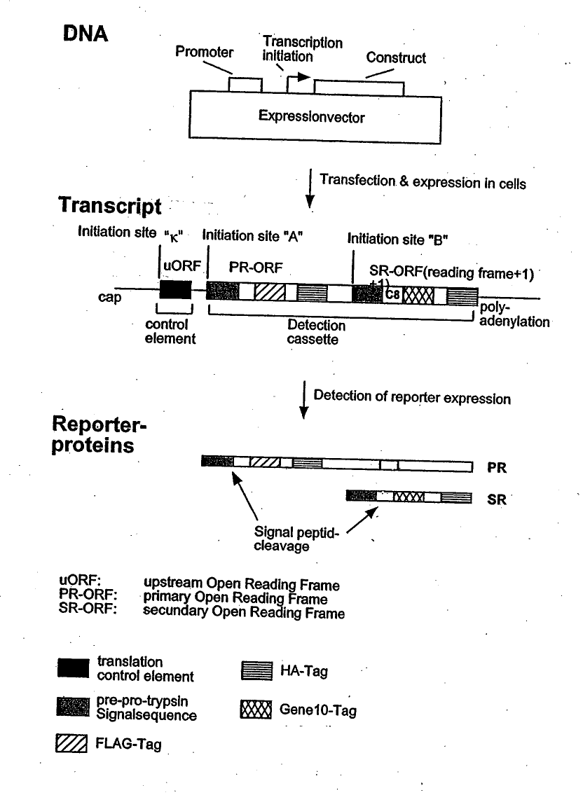

What is present is a prototype of the construct according to the invention having the following properties (cf., FIG. 1):

The eukaryotic expression vector has the following structure: about 50 base pairs of the leader sequence from β-globin mRNA. In 3′ direction, this is followed by the upstream open reading frame (uORF) of the rat C/EBP alpha gene as translational control element including the “κ” initiation site. This is followed by the detection cassette including the two reporter genes PR and SR. “A” denotes the initiation site of the PR reading frame. PR includes a signal sequence (pre-pro-trypsin) resulting in release of the primary reporter protein (PR). A FLAG sequence and a hemagglutinin (HA) epitope follow as antigenic epitope. The hemagglutinin epitope is used to immobilize the reporter protein. SR is encoded starting at initiation site (B). SR is shifted by one nucleotide towards 3′ with respect to PR. Due to the reading frame shift, no sense-SR product results when reading the ribosomes starting at “A”. In contrast, initiation at B results in a secondary reporter protein (SR) different from PR. Like PR, SR has a pre-pro-trypsin signal sequence at the N terminus. Next are a gene10 antigen epitope and an HA epitope. Both reporter constructs are terminated in 3′ overlap. The reporter protein expression from the A or B initiation site is influenced by the control element (uORF, κ).

### EXAMPLE 2

Function in Transfected Cells

The construct (FIG. 1) is transfected into cells, and the reporter protein is determined after 24 and 48 hours, respectively. Apparently, reporter proteins having the desired properties (size, secretion, antigenic properties) are produced. A similar construct, but lacking the translational control element, produces amounts of a reporter protein which differ from those produced with TCRS. Hence, the present TCRS construct is suitable for use in high-throughput screening of substances that influence translation via the control element.

### EXAMPLE 3

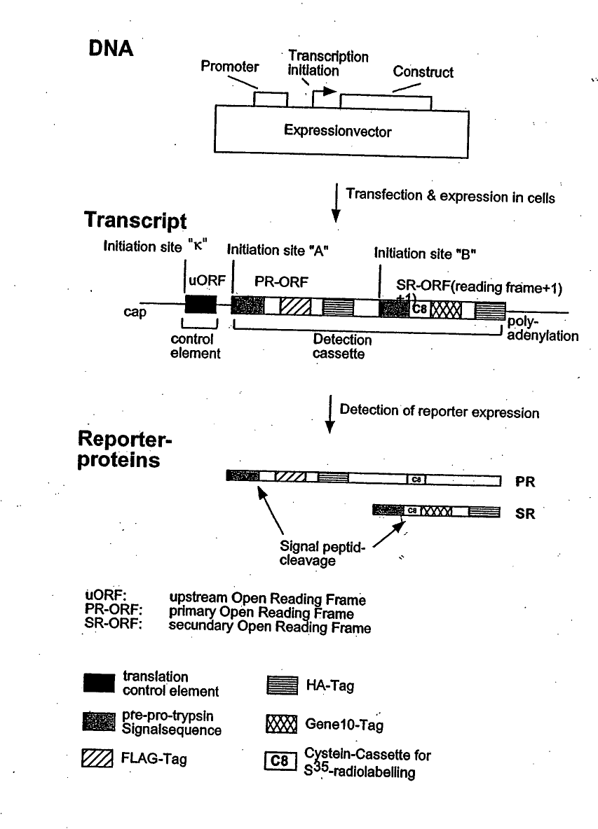

The Structure of an Additional Embodiment of a TCRS Cassette and Transcript as depicted in FIG. 2.

DNA: schematic presentation of the TCRS plasmid construct. An expression vector contains a promoter that activates transcription of the TCRS mRNA.

Transcript: schematic representation of the TCRS transcript. An upstream Open Reading Frame (uORF, black box) serves as a translational control element in front of the peptide cassettes. The uORF (or any other control element placed at this position) and the distance between uORF and translation start serves to regulate initiation from site “A” or “B”. Translation start site “B” is in another reading frame as the peptide initiated in “A”. Both peptides harbor a cytokine cassette (C8) or for radio labeling. Capture of the peptides occurs through the HA tags (both peptides) and peptides are differentiated by Flag and Gene 10 tags, respectively. Both peptides have N-terminal secretion signal sequences.

### EXAMPLE 4

Improvement of Reporter Protein Detection

To further optimize and facilitate the detection of the reporter proteins, we introduced a Cytokine-cassette containing eight csyteine residues for radioactive (Cysteine-S³⁵) labelling in each reporter protein, PR and SR, respectively (see figure). Because both reporter proteins contain the same number of cysteine residues, the relative amounts of the reporter proteins expressed from the TCRS-construct can be directly compared.

### EXAMPLE 5

Application of TCRS in an in vitro Transcription/Translation Coupled System

TCRS can be applied to in vitro screening. To examine the applicability of the TCRS-vector for the radioactive Cysteine-S³⁵ labelling, we introduced the TCRS-vector into rabbit reticulocyte translation system supplemented with S³⁵-labelled Cyteine. Both the PR- and SR reporter proteins could be visualised on a protein gel. In addition, the reporter proteins could also be immuno-precipitated from the reaction mixture and visualised on a protein gel, demonstrating the accessibility and full function of the immunotags.

Mutation in the uORF demonstrated dependence of translation products from uORF control. This confirmed that translational control of TCRS depends on the intact regulatory uORF. Another application is that drugs targeting translational control can also be screened in the presence of mutated translational control factors in vitro.

### EXAMPLE 6

Application of TCRS in Cell Lines

TCRS functions in cell lines. To examine the applicability of the TCRS in vertebrate cells we introduced the improved TCRS-vector into fibroblasts by transient transfection. After 24 hours of cultivation, the PR and SR reporter proteins could be immuno-precipitated from the culture medium and visualised on a protein separation gel, demonstrating the functional secretion and immuno-detection of the reporter proteins outside of the cell. Importantly, removal of the regulatory uORF by point side of the cell. Importantly, removal of the regulatory uORF by point mutation of its translation initiation site results in loss of translational controlled SR protein expression. Accordingly, the TCRS can be used to screen for drugs or to monitor pharmacological effects in tissue culture.

### EXAMPLE 7

Detection of Cellular Translational Control Activities by TCRS

To examine the potential of the TCRS for detection of changes in translation control activities by external supplied drugs, we treated the TCRS containing NIH-3T3 fibroblasts cells with aminopurine and rapamycin, respectively. Differential changes of the peptide levels were observed.

Treatment with translational active drugs, such as rapamycin or aminopurine, result in differential activation of distinct translation control pathways, causing a shift in reporter protein expression. Shift in reporter expression was also observed with co-transfected expression vectors encoding eucaryotic translation initiation factors eIF2or eIF4E, or mutants thereof. Reporter protein expression shifts were dependent on an intact uORF.

Hence, drugs that provoke changes in translational control in the cell can be identified and their effect qualified and quantified by use of the TCRS. Accordingly, the TCRs can also be used to screen drugs that overcome defects induced by mutation of protein components of the translation control machinery or signalling pathways.

1. A vector system for the screening of active substances, said vector system being comprised of
(a) control elements of translation, which elements are situated essentially 5′ from the detection cassette, and optionally 3′ from or within the detection cassette;
(b) a detection cassette encoding two or more protein reporter constructs in different reading frames;
(c) said reporter constructs including
(d) signal sequences for the release from the cell, and
(e) various antigenic sites allowing immobilization and differential detection of reporter proteins.

2. The vector system for the screening of active substances according to claim 1, characterized in that the vector system is coupled on derived control elements with alternative reporter genes such as GFP, luciferase for modified detection.

3. A vector system for the screening of active substances in accordance with FIG. 1 (TCRS).

4. Method of screening active substances according to claim 1, characterized in that the construct is incorporated in cells in a transient or stable fashion, the cells are subsequently exposed to substances, and the resulting reporter amounts coming from the construct are determined and compared.

5. Cell lines for use in screening active substances, said cell lines including a vector system according to claim 1.
